(19) United States
(12) Patent Application Publication
(10) Pub. No.: US 2013/0041351 A1
(43) Pub. Date: Feb. 14, 2013

(54) CLOSED WOUND DRAINAGE SYSTEM

(71) Applicant: Clinton Frederick Shahim, Sandton (ZA)

(72) Inventor: Clinton Frederick Shahim, Sandton (ZA)

(73) Assignee: Clinton Frederick Shahim, Sandton (ZA)

(21) Appl. No.: 13/392,082

(22) PCT Filed: Aug. 24, 2010
(86) PCT No.: PCT/ZA2010/000048

§ 371 Date: Mar. 6, 2012

(30) Foreign Application Priority Data
Aug. 24, 2009 (ZA) 2009/05841

(51) Int. Cl. A61M 27/00

(52) U.S. Cl. 604/543

(57) ABSTRACT

A container (10) for a closed wound drainage system which includes a screw-actuated piston (32) sealingly located in a cylinder (30) of non-circular shape to define, inside the cylinder (30), a fluid-receiving volume of variable size.

### BACKGROUND OF THE INVENTION

This invention relates to the drainage of body fluids from a closed wound.

It is important to drain fluids from a wound in an injured person or animal, for example during or after an operation. Various techniques have been proposed for this purpose—see for example U.S. Pat. No. 4,599,093, U.S. Pat. No. 4,490,331 and CA2653961.

Although, to a greater or lesser extent, devices known to the applicant which are used for draining wounds function satisfactorily they do, in the applicant's opinion, suffer from one or more disadvantages in that they can be expensive, awkward to use and unsightly.

An object of the invention is to provide a container for use in a closed wound drainage system which, at least partly addresses one or more of the aforementioned requirements.

### SUMMARY OF INVENTION

The invention provides a container for use in a closed wound drainage system which includes a body in which is formed a cylinder with a bore, a piston, in sealing contact with the bore, which partly bounds a fluid-receiving volume inside the bore, the piston being movable relative to the body thereby to vary the size of the volume, a piston rod which extends from the piston, a fluid inlet to the volume, and a screw mechanism, engaged with the piston rod, which is rotatable relative to the body, to move the piston within the bore and so cause corresponding movement of the piston.

The piston may be movable in a first direction, within the bore, to reduce pressure prevailing in the fluid-receiving volume. As the size of the volume is increased, a pressure reduction inside the volume ensues and fluid can be drawn through a suitable conduit, from a drainage location, through the inlet into the volume.

The piston rod is preferably threaded and is threadedly engaged with the screw mechanism.

The screw mechanism may include a user-actuable handle which is mounted to the body for rotational movement. The handle may be rotatable in a first direction thereby to move the piston in a first linear direction and counter-rotatable in a second rotational direction thereby to move the piston in a second linear direction.

During rotation, the handle may be held captive to the body so that it is only rotatable relative to the body and does not move meaningfully in any other way.

The screw mechanism may include at least one threaded component which acts on the threaded piston rod.

Preferably the screw mechanism includes a first elongate tubular member, a first screw formation on an inner side of the tubular member, at one end thereof, a second elongate tubular member which is externally threaded and which is engaged with the first screw formation, and a second screw formation at an end of the second tubular member on an inner side thereof which is threadedly engaged with the piston rod which, in turn, extends at least partly into a bore of the second tubular member.

The piston may be constrained against rotation while it is moved linearly within the bore. This may be achieved in any appropriate way. For example, and preferably, the piston and the bore are of complementary non-circular shapes e.g. oval shapes.

An indicator may be provided on the container. The indicator may be mounted externally and is used to provide an indication of the pressure prevailing in the fluid-receiving volume. The indicator may include a bellows, an interior of which is in communication with the fluid-receiving volume. The bellows may be positioned so that it collapses as pressure inside the volume is decreased and expands as pressure inside the volume increases.

### BRIEF DESCRIPTION OF THE DRAWINGS

The invention is further described by way of example with reference to the accompanying drawings in which:

FIG. 1 is a perspective view of a container for use in a closed wound draining system according to the invention;

FIG. 2 shows the container from one side and in cross section with a piston, inside the container, in a fully retracted position;

FIG. 3 is similar to FIG. 2 but with the piston pressed home inside a bore of the container; and

FIG. 4 is a perspective view of inner components of the container in an exploded configuration.

### DESCRIPTION OF PREFERRED EMBODIMENT

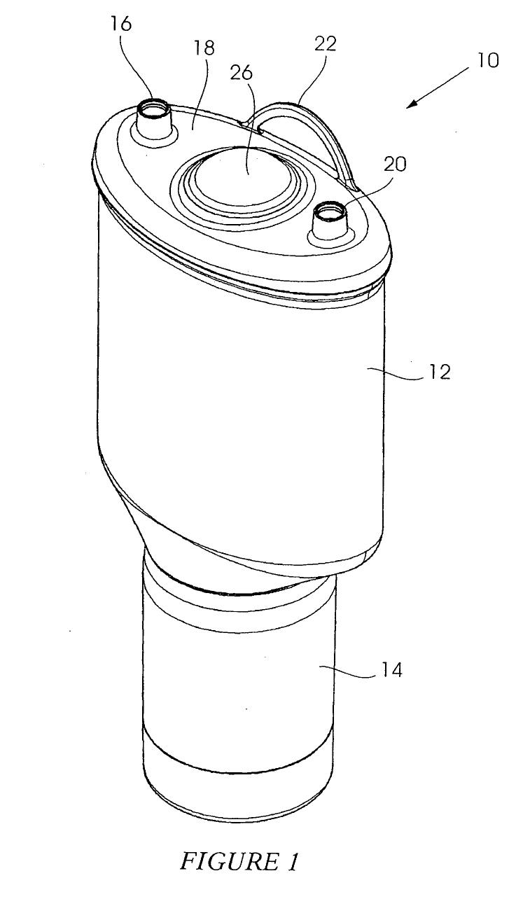

FIG. 1 of the accompanying drawings is an external view, in perspective, of a container 10 for use in a fluid drainage system. The container has a body 12 and a user-actuable handle 14 at a lower end of the body. A sealable fluid inlet 16 is positioned on one side of a lid 18. A fluid drainage outlet 20, which is also sealable, is positioned on an opposing side of the lid.

A formation 22 which allows the container to be suspended from overhead structure, not shown, is centrally located on the lid. Below the formation, extending from a circular pedestal 24, is an indicating bellows 26.

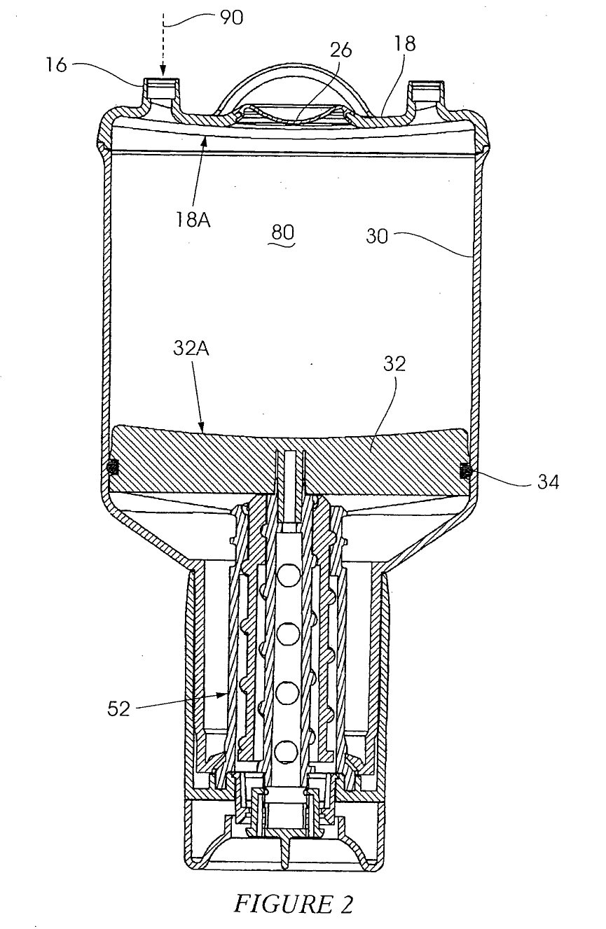

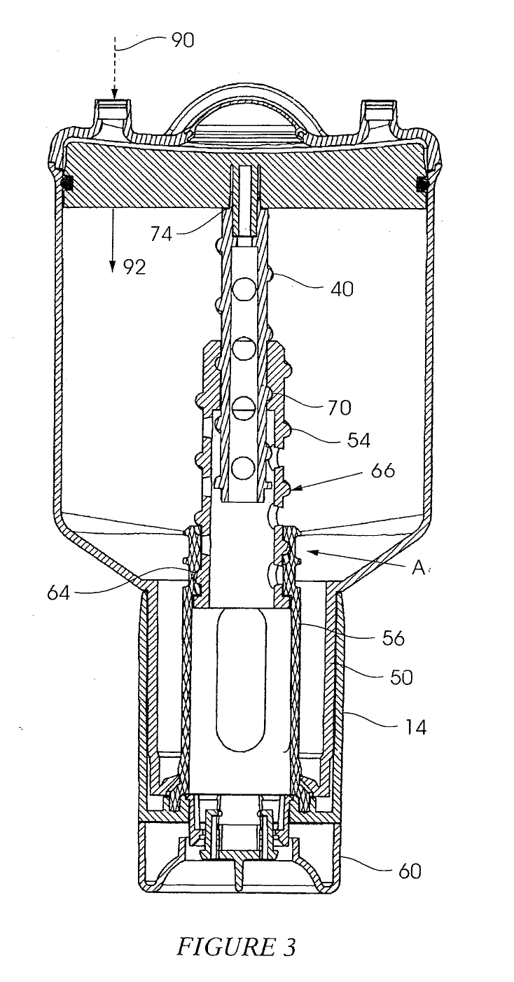

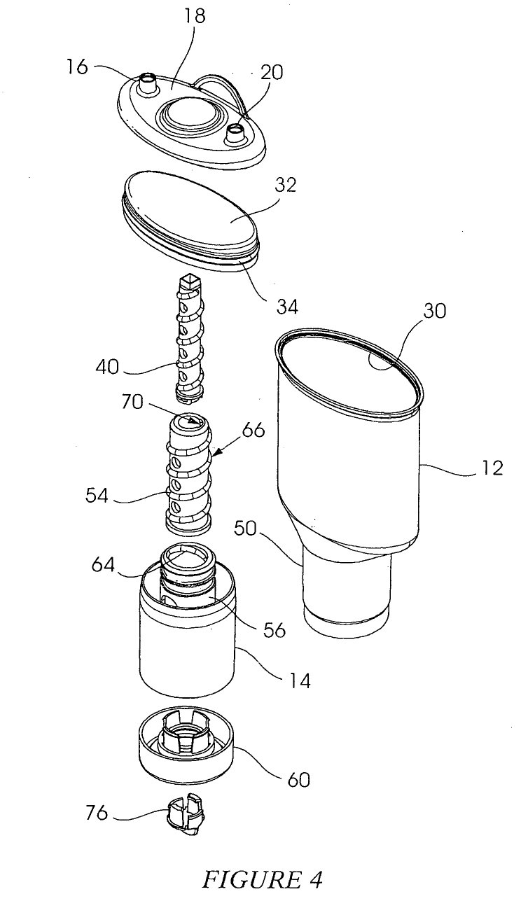

FIGS. 2 and 3 show the container 10 in cross section in different operative modes while FIG. 4 shows inner components of the container in an exploded configuration.

The body 12 is oval in shape and, internally, defines an oval-shaped cylindrical bore 30 with a smooth internal surface. A piston 32, of complementary shape to the bore, is slidingly positioned inside the bore. The piston has an external seal 34 which ensures that an intimate seal is established between the piston and the bore.

A threaded piston rod 40 projects downwardly from the piston. The thread on the rod is fairly coarse. This feature enables the piston to be moved with ease, as is described hereinafter, and also reduces the likelihood that fluid or other foreign material can meaningfully impede actuation of the piston.

The handle 14 clips onto a tubular projection 50 at a lower side of the body. A screw mechanism 52 is formed by the rotatable engagement of the handle 14 with the projection and by the threaded interengagement of the piston rod 40 with a first elongate tubular component 54 which in turn is engaged with a second elongate tubular component 56.

A cap 60 is clipped onto a lower side of the handle 14. The second elongate tubular component 56 is centrally positioned inside the handle 14 and, at an upper end has a short threaded section 64. The first component 54 has a thread 66 over its length which is engaged with the threaded section 64. On an inner side, at an upper end, the first component has a threaded portion 70 with which the externally threaded piston rod is threadedly engaged.

An upper end of the piston rod 40 bears against and is connected to complementary formations 74 at what, in use, is a lower side of the piston.

The handle is secured to the projection 50 by means of a clip device 76.

The tubular component 56, at a lower end, is held captive to the handle and thus is rotatable together with the handle which, in turn, is held captive to the tubular projection 50. Thus, upon rotation of the handle in one direction the tubular section 56 is also rotated, in the same direction. The threaded section 64 then rides over the thread 66 and causes the first elongate tubular component to be elevated relative to the threaded section 64. When a limiting position is reached the first elongate tubular component 54 is locked to the component 56 and can no longer be threadedly advanced relative to the threaded section. This locking configuration, designated A, is shown in FIG. 3. The tubular component 54 is then rotated and as a consequence, in a manner which is similar to what has been described, the piston rod is advanced along the threaded portion 70 to project from the tubular component 54 as is shown in FIG. 3.

The cylindrical bore 30 and the piston 32 are of complementary oval shape. The piston cannot therefore rotate inside the bore and, consequently, rotational movement of the handle 14 is directly translated into linear movement of the piston, without play or lost movement occurring.

During the aforementioned process a fluid-receiving volume 80 which is formed inside the bore between opposing surfaces of the lid 18 and the piston 32 is varied in size. The piston can be moved between a lower extreme position at which the volume is at a maximum, see FIG. 2, and an upper extreme position at which the volume is at a minimum, see FIG. 3.

The indicator bellows 26 is made from a flexible plastic material and is exposed to the volume 80.

The fluid inlet 16 and the fluid outlet 20 can be sealed by means of suitable caps, not shown.

In use of the container an elongate flexible drainage conduit indicated notionally by means of a dotted line 90 in FIG. 2 is connected to the inlet 16. A free end of the conduit is positioned, as is known in the art, in a body cavity from which fluid is to be drained. At this stage the piston is in the limiting position shown in FIG. 3 i.e. with the volume 80 as small as possible. A lower inner surface 18A of the lid 18 has a complementary shape to an upper opposing surface 32A of the piston 32 and thus, for practical purposes, the volume is effectively reduced to zero. This helps to maximize the fluid receiving capability of the volume 80.

The handle 14 is then rotated in the appropriate direction to cause the piston to move, inside the bore, in the direction of an arrow 92. In so doing the size of the volume 80 is increased. As the connections to, and in, the container are essentially airtight the volume increase translates into a reduction in the pressure prevailing in the volume to a level which is slightly below atmospheric pressure. Fluid in the body cavity is then expelled by atmospheric pressure action into the conduit 90 and flows into the volume 80 where it is collected.

The bellows 26 provides a visual indication of the prevailing pressure in the volume 80. If the pressure in the volume is essentially at atmospheric level then the bellows extends to the position shown in FIG. 3 under the inherent resilience of the material from which the bellows is made. On the other hand as the pressure in the volume is decreased the atmospheric pressure acting on an outer side of the bellows causes the bellows to collapse to a greater or lesser extent, as is shown for example in FIG. 2.

If the bellows at any time indicates that the pressure inside the volume 80 has reverted to atmospheric then the handle 14 is rotated appropriately to reduce the pressure and so ensure that drainage from the wound site continues to take place.

The body of the container can include a sight glass or can be made from an appropriate transparent material so that a visual indication is available to a user of the content of the volume.

When the container is no longer to be used the conduit 90 is detached from the inlet which is then sealed, as is shown in FIG. 1, and the container is then disposed of in an acceptable manner. The outlet 20 is provided so that, if required, fluid can be extracted from the volume 80.

An optional feature is to engage a flap valve (not shown) with the inlet 16. The flap valve acts as a non-return valve and ensures that fluid cannot flow from the volume into the conduit.

1-7. (canceled)

8. A container (10) for use in a closed wound drainage system which includes a body (12) in which is formed a cylinder with a bore (30), a piston (32), in sealing contact (34) with the bore, which partly bounds a fluid-receiving volume (80) inside the bore, the piston being movable relative to the body, a piston rod (40) which extends from the piston, and a fluid inlet (16) to the volume, characterized in that the piston and the bore are of complementary non-circular shapes so that the piston is constrained against rotation while it is moved linearly within the bore, in that the container includes a screw mechanism (52), threadedly engaged (70) with the piston rod (40), which includes a user-actuable handle (14) which is held captive (50) to the body and which is mounted to the body only for rotational movement, relative to the body, in a first direction thereby to move the piston (32) in a first linear direction or in a second rotational direction thereby to move the piston in a second linear direction, thereby to vary the size of the volume (80) and in that the piston rod (40) is located inside the bore (30) and the handle (14).

9. A container according to claim 8 characterized in that the screw mechanism (52) includes a first elongate tubular member (54), a first screw formation (66) on an inner side of the first tubular member, at one end thereof, a second elongate tubular member (56) which is externally threaded (64) and which is engaged with the first screw formation, and a second screw formation (70) at an end of the second tubular member on an inner side thereof which is threadedly engaged with the piston rod (40) which, in turn, extends at least partly into a bore of the second tubular member (56).

10. A container according to claim 9 characterized in that it includes a bellows (26), an interior of which is in communication with the fluid-receiving volume (80) so that it collapses as pressure inside the volume is decreased and expands as pressure inside the volume increases.

11. A container according to claim 8 characterized in that it includes a bellows (26), an interior of which is in communication with the fluid-receiving volume (80) so that it collapses as pressure inside the volume is decreased and expands as pressure inside the volume increases.
